(19) United States
(12) Patent Application Publication
(10) Pub. No.: US 2009/0118521 A1
(43) Pub. Date: May 7, 2009

(54) NANOENGINEERED ORGANIC NONLINEAR OPTICAL GLASSES

(75) Inventors: Kwan-Yue Jen, Kenmore, WA (US); Jingdong Luo, Seattle, WA (US); Tae-Dong Kim, Seattle, WA (US); Baoquan Chen, Bothell, WA (US); Jae-Wook Kang, Gyeongsangnam-do (KR)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: WASHINGTON, UNIVERSITY OF, Seattle, WA (US)

(21) Appl. No.: 11/462,343

(22) Filed: Aug. 3, 2006

Related U.S. Application Data
(63) Continuation-in-part of application No. 11/335,840, filed on Jan. 18, 2006, now abandoned.
(60) Provisional application No. 60/645,309, filed on Jan. 18, 2005.
(60) Provisional application No. 60/646,241, filed on Jan. 21, 2005.

(51) Int. Cl. C07D 333/32; C07D 307/28; B32B 9/00; B01J 19/08; G01B 9/02

(52) U.S. Cl. 549/4; 549/214; 428/220; 427/457; 356/450; 356/256

(57) ABSTRACT

Nonlinear optically active compounds having film-forming properties, films including the compounds, methods for making the compounds and films, and electro-optic devices including the films and compounds.

### CROSS-REFERENCES TO RELATED APPLICATIONS

This application is a continuation-in-part of U.S. application Ser. No. 11/335,840, filed Jan. 18, 2006, which claims the benefit of U.S. Provisional Application No. 60/645,309, filed Jan. 18, 2005, and U.S. Provisional Application No. 60/646,241, filed Jan. 21, 2005, each expressly incorporated herein by reference in its entirety.

### STATEMENT OF GOVERNMENT LICENSE RIGHTS

This invention was made with government support under Contact Number N00014-04-1-0094, awarded by the United States Navy. The government has certain rights in the invention.

### BACKGROUND OF THE INVENTION

Electrical signals can be encoded onto fiber-optic transmissions by electro-optic modulators. These modulators include electro-optic materials having highly polarizable electrons. When these materials are subject to an electric field, their polarization changes dramatically resulting in an increase in the index of refraction of the material and an accompanying decrease in the velocity of light traveling through the material. This electric field-dependent index of refraction can be used to encode electric signals onto optical signals. Uses include, for example, switching optical signals and steering light beams.

A variety of electro-optic materials have been utilized for use in electro-optic devices. Among these materials are inorganic materials such as lithium niobate, semiconductor materials such as gallium arsenide, organic crystalline materials, and electrically poled polymer films that include organic chromophores. A review of nonlinear optical materials is provided in L. Dalton, “Nonlinear Optical Materials,” Kirk-Othmer Encyclopedia of Chemical Technology, 4^{th} ed., Vol. 17 John Wiley & Sons, New York, pp. 288-302 (1995).

In contrast to inorganic materials in which polar optical lattice vibrations diminish effectiveness, the optical properties of organic nonlinear optical materials depend primarily on the hyperpolarizability of their electrons without a significant adverse contribution from the lattice polarizability. Thus, organic nonlinear optical materials offer advantages for ultrafast electro-optic modulation and switching.

Lithium niobate, a common material currently utilized in electro-optic devices, has an electro-optic coefficient of about 35 pm/V resulting in a typical drive voltage of about 5 volts. Drive voltage (V_{π}) refers to the voltage that produces a π phase shift of light. Lithium niobate has a high dielectric constant (ε=28), which results in a mismatch of electrical and optical waves propagating in the material. The mismatch necessitates a short interaction length, which makes drive voltage reduction through increasing device length unfeasible, thereby limiting the device's bandwidth. Recent lithium niobate modulators have been demonstrated to operate at a bandwidth of over 70 GHz.

Electro-optic poled polymers have also been utilized as modulating materials. Their advantages include their applicability to thin-film waveguiding structures, which are relatively easily fabricated and compatible with existing microelectronic processing. These polymers incorporate organic nonlinear optically active molecules to effect modulation. Because organic materials have low dielectric constants and satisfy the condition that n²=ε, where n is the index of refraction and ε is the dielectric constant, organic electro-optic will have wide bandwidths. The dielectric constant of these materials (ε=2.5-4) relatively closely matches the propagating electrical and optical waves, which provides for a drive voltage in the range of about 1-2 volts and a bandwidth greater than 100 GHz.

Advantages of organic nonlinear optical materials include a bandwidth in excess of 100 GHz/cm device and ease of integration with semiconductor devices. See L. Dalton et al., “Synthesis and Processing of Improved Organic Second-Order Nonlinear Optical Materials for Applications in Photonics,” Chemistry of Materials, Vol. 7, No. 6, pp. 1060-1081 (1995). In contrast to inorganic materials, these organic materials can be systematically modified to improve electro-optic activity by the design and development of new organic materials and by the development of improved processing methods. See L. Dalton et al., “The Role of London Forces in Defining Noncentrosymmetric Order of High Dipole Moment-High Hyperpolarizability Chromophores in Electrically Poled Polymeric Films,” Proceedings of the National Academy of Sciences USA, Vol. 94, pp. 4842-4847 (1997).

For an organic nonlinear optical material to be suitable for electro-optic applications, the material should have a large molecular optical nonlinearity, referred to as hyperpolarizability (β), and a large dipole moment (μ). A common figure of merit used to compare materials is the value μβ. Organic materials having μβ values greater than about 15,000×10⁻⁴⁸ esu that also satisfy the desired thermal and chemical stability and low optical loss at operating wavelengths have only recently been prepared. See Dalton et al., “New Class of High Hyperpolarizability Organic Chromophores and Process for Synthesizing the Same,” WO 00/09613. However, materials characterized as having such large μβ values suffer from large intermolecular electrostatic interactions that lead to intermolecular aggregation resulting in light scattering and unacceptably high values of optical loss. A chromophore's optical nonlinearity (μβ) can be measured as described in Dalton et al., “Importance of Intermolecular Interactions in the Nonlinear Optical Properties of Poled Polymers,” Applied Physics Letters, Vol. 76, No. 11, pp. 1368-1370 (2000). A chromophore's electro-optic coefficient (r₃₃) can be measured in a polymer matrix using attenuated total reflection (ATR) technique at telecommunication wavelengths of 1.3 or 1.55 μm. A representative method for measuring the electro-optic coefficient is described in Dalton et al., “Importance of Intermolecular Interactions in the Nonlinear Optical Properties of Poled Polymers,” Applied Physics Letters, Vol. 76, No. 11, pp. 1368-1370 (2000).

Many molecules can be prepared having high hyperpolarizability values, however their utility in electro-optic devices is often diminished by the inability to incorporate these molecules into a host material with sufficient noncentrosymmetric molecular alignment to provide a device with acceptable electro-optic activity. Molecules with high hyperpolarizability typically exhibit strong dipole-dipole interactions in solution or other host material that makes it difficult to achieve a high degree of noncentrosymmetric order without minimizing undesirable spatially anisotropic intermolecular electrostatic interactions.

Chromophore performance is dependent on chromophore shape. See Dalton et al., “Low (Sub-1-Volt) Halfwave Voltage Polymeric Electro-optic Modulators Achieved by Controlling Chromophore Shape,” Science, Vol. 288, pp. 119-122 (2000).

Chemical, thermal, and photochemical stabilities are imparted to the chromophores through their chemical structure and substituent choice. For example, in certain embodiments, the chromophore's active hydrogens are substituted with groups (e.g., alkyl, fluorine) to impart increased stability to the chromophore.

Thus, the effectiveness of organic nonlinear optical materials having high hyperpolarizability and large dipole moments can be limited by the tendency of these materials to aggregate when processed into electro-optic devices. The result is a loss of optical nonlinearity. Accordingly, improved nonlinear optically active materials having large hyperpolarizabilities and large dipole moments and that, when employed in electro-optic devices, exhibit large electro-optic coefficients may be advantageous for many applications.

For the fabrication of practical electro-optical (E-O) devices, critical material requirements, such as large E-O coefficients, high stability (thermal, chemical, photochemical, and mechanical), and low optical loss, need to be simultaneously optimized. In the past decade, a large number of highly active nonlinear optical (NLO) chromophores have been synthesized, and some of these exhibit very large macroscopic optical nonlinearities in high electric field poled guest/host polymers. To maintain a stable dipole alignment, it is a common practice to utilize either high glass transition temperature (T_{g}) polymers with NLO chromophores as side chains or crosslinkable polymers with NLO chromophores that could be locked in the polymer network. However, it is difficult to achieve both large macroscopic nonlinearities and good dipole alignment stability in the same system. This is due to strong intermolecular electrostatic interactions among high dipole moment chromophores and high-temperature aromatic-containing polymers, such as polyimides and polyquinolines that tend to form aggregates. The large void-containing dendritic structures may provide an attractive solution to this critical issue because the dendrons can effectively decrease the interactions among chromophores due to the steric effect. Furthermore, these materials are monodisperse, well-defined, and easily purifiable compared to polymers that are made by the conventional synthetic approaches.

### SUMMARY OF THE INVENTION

The present invention provides compounds having film-forming properties, films including the compounds, methods for making the compounds and films, and devices including the films and compounds.

In one aspect, the invention provides compounds having film-forming properties. In one embodiment, the compounds have a π-electron donor group electronically conjugated to a π-electron acceptor group through π-electron bridge group, and have the formula:

D-π₁-B-π₂-A

wherein D is a π-electron donor group, B is a π-electron bridge group, A is a π-electron acceptor group, π₁ is a π bridge electronically conjugating D to B, π₂ is a π bridge electronically conjugating B to A, wherein π₁ and π₂ may each be present or absent, and wherein one or more of the donor, bridge, and acceptor groups are substituted with one or more substituents having steric bulk.

In another aspect, the present invention provides a film composed of the compounds having film-forming properties. In one embodiment, the film includes only a compound having film-forming properties.

In one embodiment, the invention provides a method for forming an at least partially aligned chromophore film, comprising:

(a) depositing a compound onto a substrate to provide a film, wherein the film consists essentially of a compound, having a π-electron donor group electronically conjugated to a π-electron acceptor group through π-electron bridge group, the compound having the formula:

D-π₁-B-π₂-A

wherein D is a π-electron donor group, B is a π-electron bridge group, A is a π-electron acceptor group, π₁ is a π bridge electronically conjugating D to B, π₂ is a π bridge electronically conjugating B to A, wherein π₁ and π₂ may each be present or absent;

(b) subjecting the film to a temperature equal to or greater than the glass transition temperature of the compound;

(c) applying an aligning force to the film; and

(d) reducing the temperature of the film below the glass transition temperature of the compound to provide a film comprising an at least partially aligned chromophore film.

In another aspect of the invention, devices that include the compounds and films are provided.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates three representative compounds of the invention;

FIG. 2 illustrates the synthesis of a representative compound of the invention;

FIG. 3 is a differential scanning calorimetric analysis of a representative compound of the invention;

FIG. 4 illustrates the synthesis of a representative compound of the invention;

FIG. 5 illustrates the synthesis of a representative compound of the invention;

FIG. 6 illustrates representative donor groups useful in making compounds of the invention;

FIG. 7 illustrates representative acceptor groups useful in making compounds of the invention;

FIG. 8 illustrates representative bridge groups useful in making compounds of the invention;

FIG. 9A illustrates a representative family of compounds useful in making compounds of the invention;

FIG. 9B illustrates the synthesis of a representative compound of the invention;

FIG. 10 is a differential scanning calorimetric analysis of a representative compound of the invention;

FIG. 11 illustrates a representative family of compounds of the invention;

FIG. 12 illustrates the synthesis of a representative compound of the invention; and

FIG. 13 illustrates the synthesis of a representative compound of the invention.

### DETAILED DESCRIPTION OF THE INVENTION

The present invention provides compounds having film-forming properties, films including the compounds, methods for making the compounds and films, and devices including the compounds and films.

In one aspect, the invention provides compounds having film-forming properties.

The compounds are nonlinear optically active compounds. The compounds of the invention include a π-electron donor group (D) electronically conjugated to a π-electron acceptor group (A) through π-electron bridge group (B).

The compounds of the invention have the general formula:

D-π₁-B-π₂-A

wherein D is a π-electron donor group, B is a π-electron bridge group, A is a π-electron acceptor group, π₁ is a π bridge electronically conjugating D to B, π₂ is a π bridge electronically conjugating B to A, wherein π₁ and π₂ may each be present or absent.

An “acceptor” (represented by A) is an atom or group of atoms with high electron affinity relative to a donor such that, when the acceptor is conjugated to a donor through a π-electron bridge, electron density is transferred from the acceptor to the donor.

A “bridge” (represented by B) is an atom or group of atoms that electronically conjugates the donor to the acceptor such that, when the acceptor is conjugated to the donor, electron density is transferred from the acceptor to the donor.

Representative donor, acceptor, and bridge groups known to those skilled in the art are described in U.S. Pat. Nos. 6,067,186; 6,090,332; 5,708,178; and 5,290,630.

The compounds of the invention have film-forming properties. As used herein, the term “film-forming properties” refers to the ability of the compound to form a film composed of only the compound itself. A compound has film-forming properties when the compound is capable of forming a film by the spin casting (or spin coating) method to provide a film that can be poled with an electric field to provide a film having poling-induced alignment of at least a portion of the compounds making the film to provide a film for which its electro-optic activity can be measured.

Prior art in the field demonstrates the use of chromophores incorporated 25 wt % or less in an inert polymer host. The polymer host provides a matrix that can be softened and hardened to allow for electrostatic poling of the guest chromophore molecules. Typical organic electro-optic chromophores do not form a glassy film when deposited without a host, and thus the need for a host, such as a polymer, arises. The present invention describes chromophores that form a free-standing solid film (or glass) and thus require no additional polymer host to be effectively incorporated into electro-optic devices.

The compounds of the invention have the formula noted above in which one or more of the donor, bridge, and acceptor groups is substituted with one or more substituents having steric bulk. In the context of the present invention, substituents having steric bulk are substituents that improve E-O activity of the compound (or film formed from the compound) compared to the corresponding compound lacking such substituents having steric bulk. Substituents having steric bulk include alkyl substituted silyl groups, substituted or unsubstituted alkyl groups, substituted or unsubstituted heteroalkyl groups, substituted or unsubstituted aryl groups, and substituted or unsubstituted heteroaryl groups.

Suitable alkyl substituted silyl groups include t-butyldimethylsilyl and trialkylsilyl groups. Suitable alkyl groups include branched or straight chain alkyl groups. In one embodiment, the branched or straight chain alkyl groups include alkyl groups having three or more carbon atoms. Suitable heteroalkyl groups include branched or straight chain heteroalkyl groups. In one embodiment, the branched or straight chain heteroalkyl groups include heteroalkyl groups having three or more carbon atoms. Suitable aryl groups include substituted phenyl groups. Suitable heteroaryl group include substituted thiophene groups.

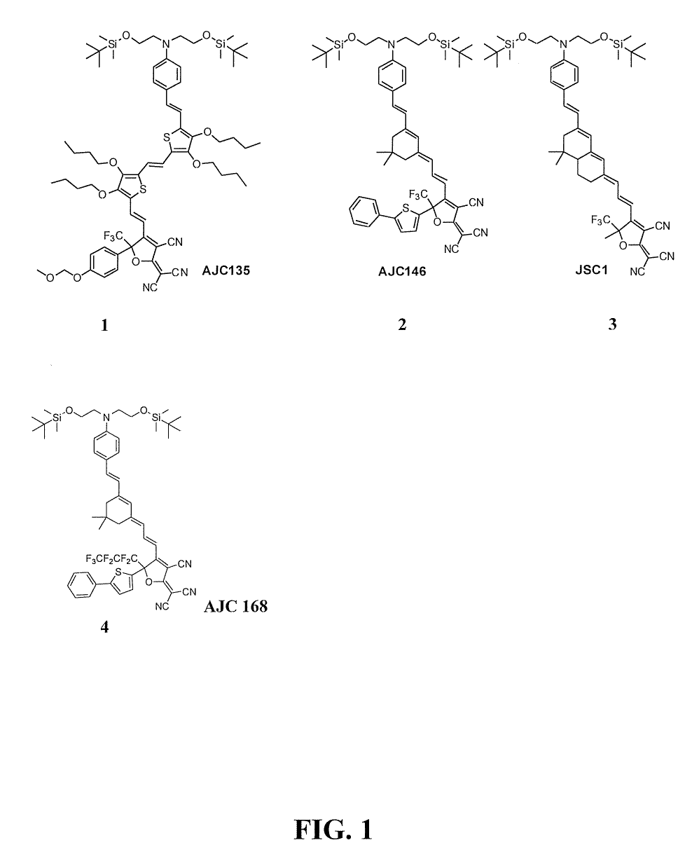

The present invention provides high μβ compounds having modified shape to provide better site-isolation and compatibility with host matrix. In certain embodiments, compounds of the invention (e.g., FIG. 1, Compounds 1, 2, 3, and 4) exhibit excellent film-forming ability by themselves. In most of these cases, the backbone of the chromophores along the donor-bridge-acceptor axis provides the path for efficient polarization while their periphery is functionalized with some nonplanar and bulky substituents to adjust the size of these chromophores for preventing them from forming dipole pair or “crystallization.”

The preparations of representative compounds of the invention are described in Examples 1-4, and illustrated in FIGS. 1-3, 5, 9B, 12, and 13.

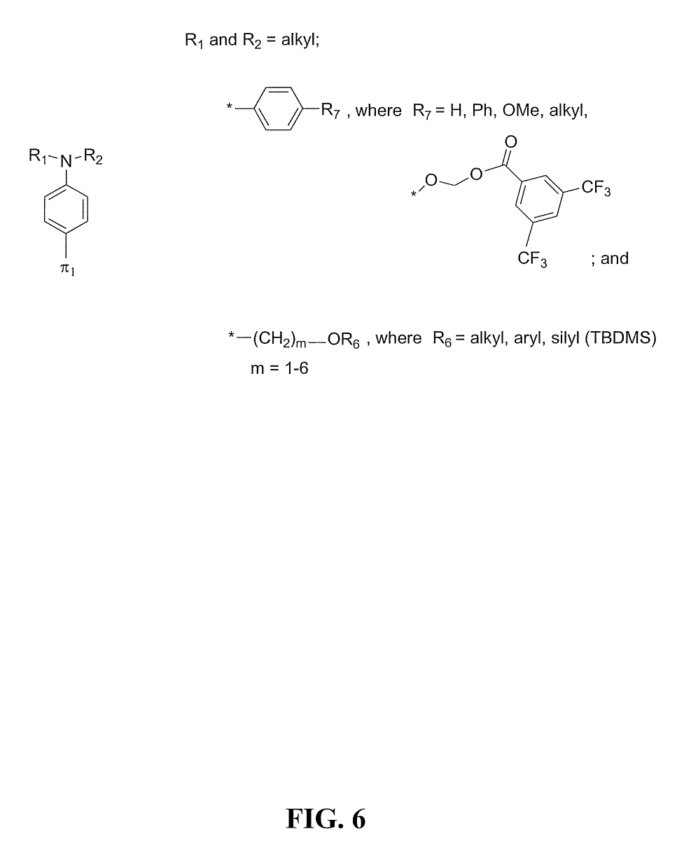

Donor groups useful in making representative compounds of the invention are illustrated in FIG. 6.

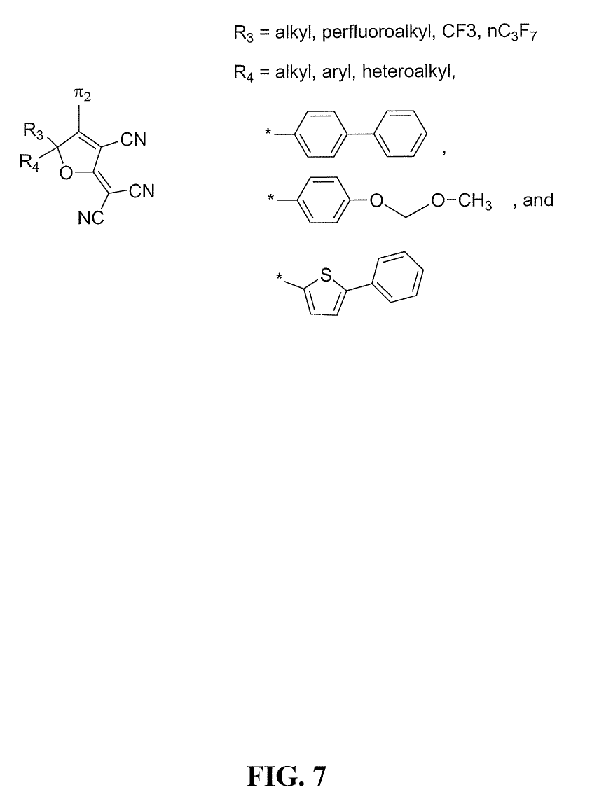

Bridge groups useful in making representative compounds of the invention are illustrated in FIG. 7.

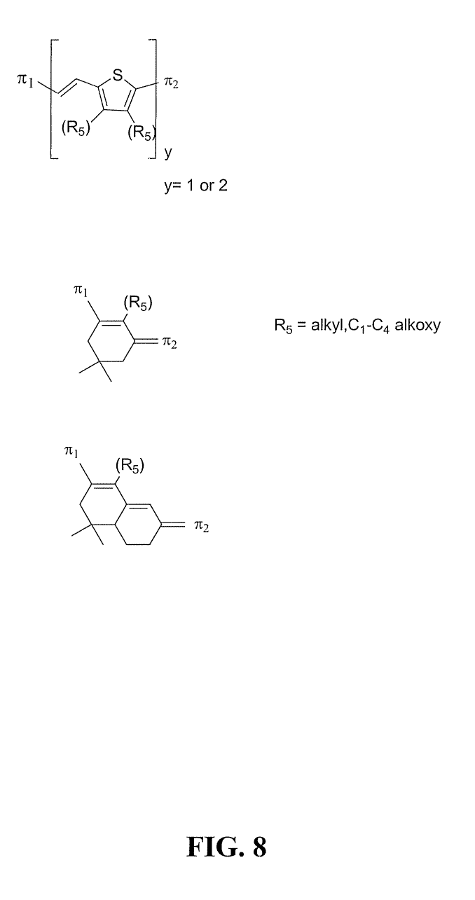

Acceptor groups useful in making representative compounds of the invention are illustrated in FIG. 8.

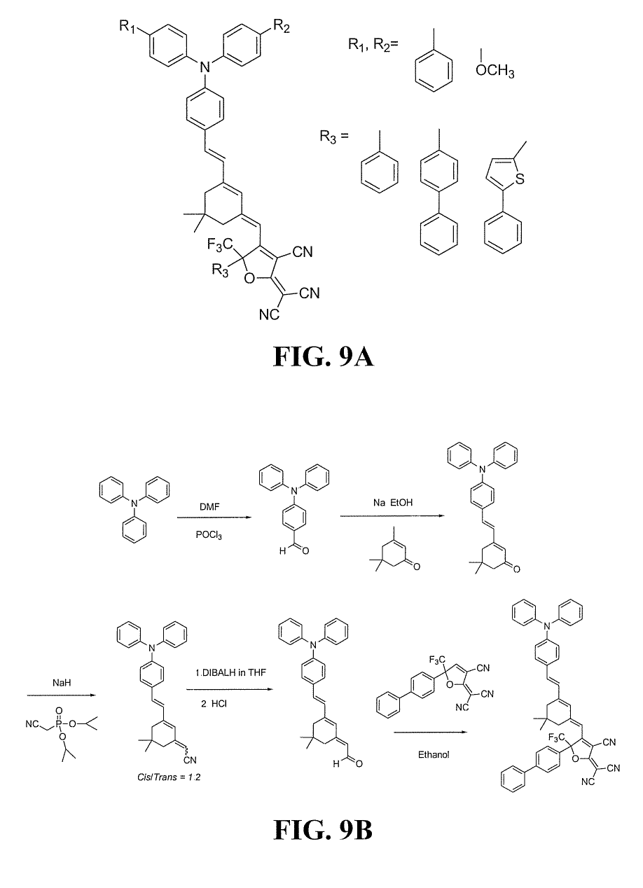

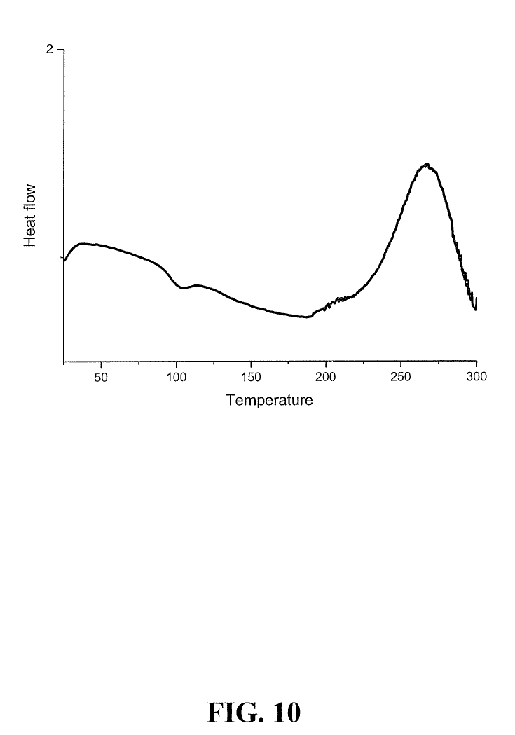

The general form for a particular family of representative chromophores is illustrated in FIG. 9A and the synthetic route to a particular species of the family is illustrated in FIG. 9B. A differential scanning calorimetry (DSC) scan of a representative compound from the FIG. 9 family is illustrated in FIG. 10.

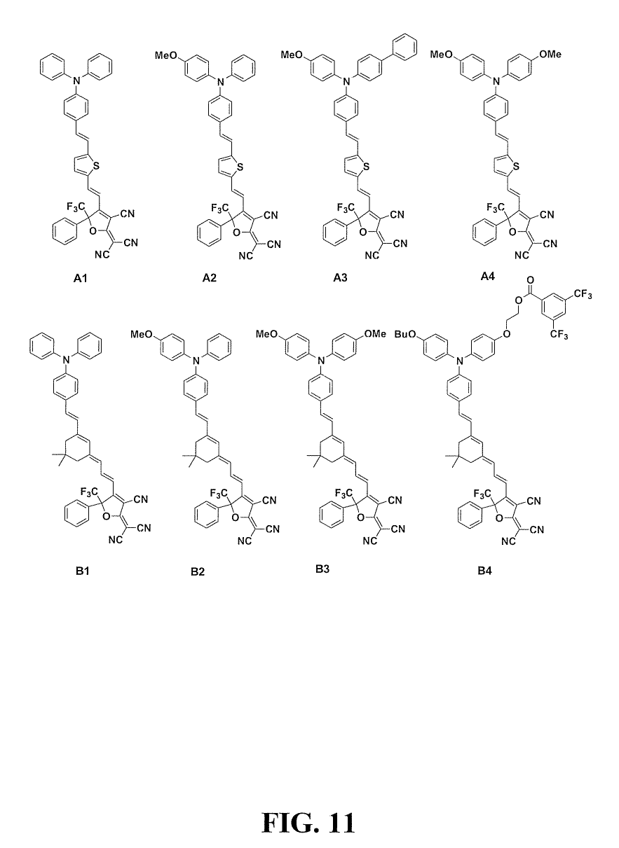

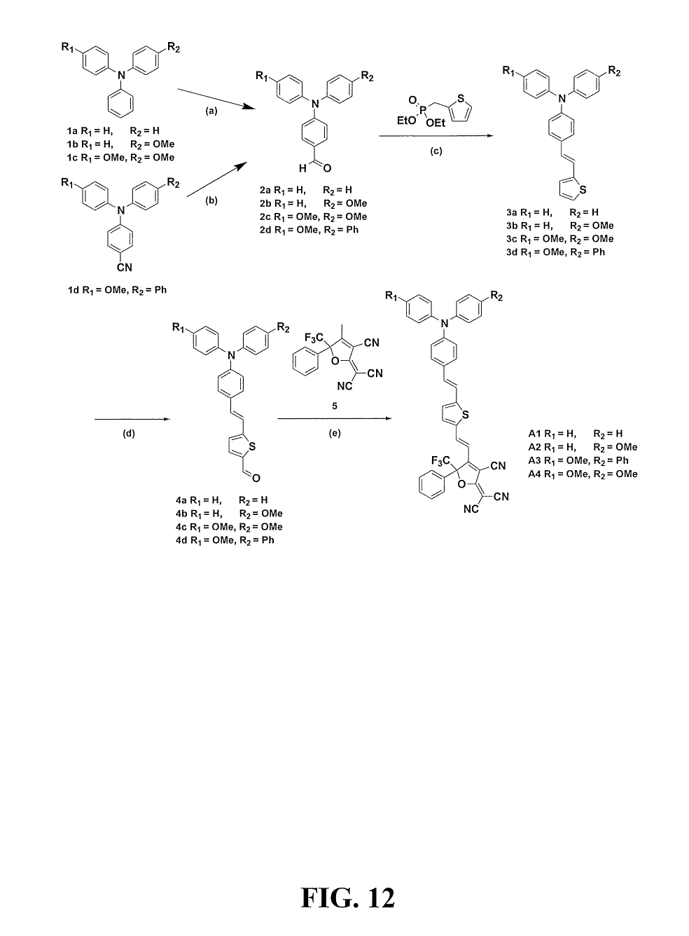

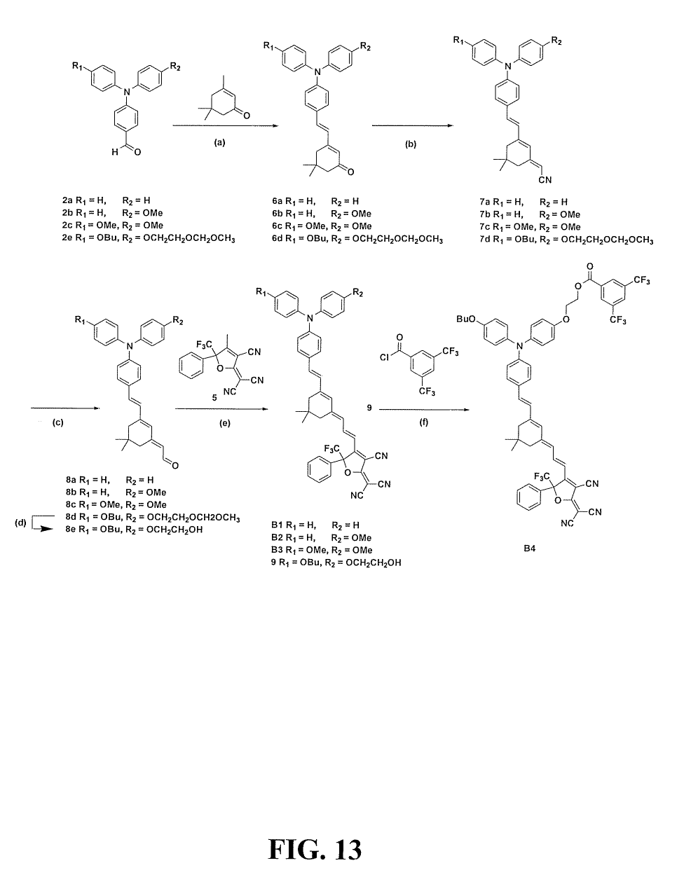

FIG. 11 represents a representative family of glass forming chromophore compounds of the invention. The synthesis of representative groups A1-A4 is illustrated in FIG. 12. The synthesis of representative groups B1-B4 is illustrated in FIG. 13. In the group of compounds illustrated in FIG. 11, a range of triarylamino donors in two series of nonlinear optical chromophores, A and B, has been systematically investigated. The π-donor strength of the triphenylamino group can be tuned and enhanced through the incorporation of donating alkoxy groups para to the nitrogen on the phenyl rings. All of these chromophores show amorphous behavior by DSC and can form monolithic thin films. With the aid of two donating methoxy substituents on the aromatic donor, both A4 and B3 exhibit significantly improved thermal stability while maintaining a high E-O effect. A shape modification with a perfluoro-aromatic dendron anchored at the donor in B4 can further improve the thermal stability and poling efficiency, leading to the highest T_{d} of 237° C. and the largest E-O coefficient (r₃₃) of 169 pm/V.

In another aspect of the invention, compounds having the following formula are provided:

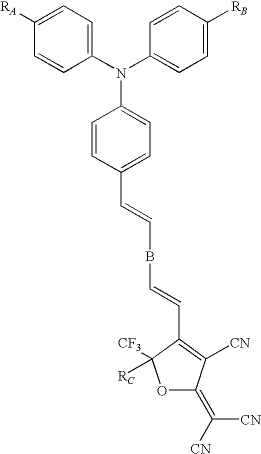

wherein B is selected from

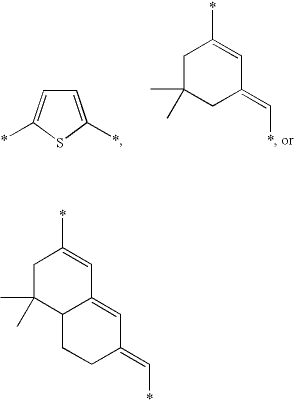

where * indicates the point of attachment to the π-bridge components of the compound;

R_{A} and R_{B} are independently selected from hydrogen, C₁-C₄ alkoxy, substituted and unsubstituted phenyl, and

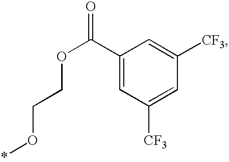

where * indicates the point of attachment to the donor phenyl groups; and

R_{C} is selected from substituted and unsubstituted phenyl, substituted and unsubstituted biphenyl, and substituted and unsubstituted thiophenyl.

The compounds of the invention are characterized as having high electro-optic coefficients; large hyperpolarizability; large dipole moments; chemical, thermal, electrochemical, and photochemical stability; low absorption at operating wavelengths (e.g., 1.3 and 1.55 μm); and suitable solubility in spin casting solvents.

Nonlinear optical activity of an organic material depends mainly on the material's hyperpolarizability (β). A measure of a compound's nonlinearity is μβ, where μ is the compound's dipole moment. A compound's optical nonlinearity (μβ) can be measured as described in Dalton et al., “Importance of Intermolecular Interactions in the Nonlinear Optical Properties of Poled Polymers”, Applied Physics Letters, Vol. 76, No. 11, pp. 1368-1370 (2000).

A material's electro-optic coefficient (r₃₃) can be measured using attenuated total reflection (ATR) technique at telecommunication wavelengths of 1.3 or 1.55 μm. A representative method for measuring the electro-optic coefficient is described in Dalton et al., “Importance of Intermolecular Interactions in the Nonlinear Optical Properties of Poled Polymers”, Applied Physics Letters, Vol. 76, No. 11, pp. 1368-1370 (2000).

In another aspect, the present invention provides films formed from the compounds. In this aspect, the present invention provides new materials having high chromophore number density and that can be processed to achieve high poling efficiency. The films of the invention are similarly characterized as having high electro-optic coefficients; large hyperpolarizability; large dipole moments; chemical, thermal, electrochemical, and photochemical stability; low absorption at operating wavelengths (e.g., 1.3 and 1.55 μm).

In one embodiment, the films include only a nonlinear optically active compound of the invention. In such embodiments, the film is a molecular film, molecular glass, or monolithic glass composed of a compound of the invention. As used herein, the terms “molecular film,” “molecular glass,” and “monolithic glass” are used interchangeably and refer to films or glasses that include only a compound of the invention (i.e., the film or glass includes no other material and does not include a host or matrix, such as a polymer host). In one embodiment, the molecular film includes more than one compound of the invention (i.e., mixtures including two or more representative compounds of the invention).

Molecular glass materials of the invention formed from the compounds of the invention exhibit high electro-optic coefficients. For example, the E-O activity of the monolithic glass of Compounds 1 and 2 exhibit r₃₃ value tip to 70 and 150 pm/V, respectively, when poled with relatively low poling fields from about 25 to about 40 V/μm due to their high conductivity at temperature around T_{g}. In spite of this limitation, they have shown great potential for achieving very high r₃₃ values. Thus, the present invention provides organic NLO materials having ultrahigh nonlinearity.

The films of the invention can be prepared by spin coating (or spin casting) methods. Briefly, a solution of the compound in a suitable spin casting solvent (e.g., cyclopentanone) (e.g., about 20% solid content filtered through 0.2 nm PTFE syringe filter) is spin coated onto half-etched ITO glass substrates at a spread of 500 rpm and spin rate of 1000 rpm. See, Mortazavi, M. A., et al., Appl. Phys. B. 53:287, 1991. The resulting films have good optical quality and micron thickness (e.g., 1.2 μm). The film may be hard-baked under vacuum at 65° C. for more than 12 hours to ensure the removal of the residual solvent. To perform high electric field poling, a thin layer of gold can be sputtered onto the film as the top electrode. Contact poling of the film at 90° C. for 5 minutes with a DC electric field of 1.0 MV/cm under nitrogen atmosphere provides the poled film having acentrically-aligned compounds (see, Ma, H., et al., Chem. Mater. 11:2218, 1999). The E-O coefficient (r₃₃) value is measured using the simple reflection technique at 1.3 μm communication wavelength (see, Teng, C. C., et al., Appl. Phys. Lett. 56(18):30, 1990. The E-O activity of the poled films of Compounds 1 and 2 exhibited r₃₃ value up to 70 and 150 pm/V, respectively. These poled films retained their original r₃₃ values for a prolonged period demonstrating their temporal stability.

Representative amorphous chromophores are soluble in chloroform, cyclopentanone, 1,1,2-trichloroethane, and THF. Pinhole free thin films can be prepared by spin coating directly from the 1,1,2-trichloroethane or chloroform solutions. The film surfaces are highly uniform according to atomic force microscopy (AFM) images, with about 0.5 nm of root-mean-squared roughness. The formation of molecular glasses and thermal transition properties can also be studied by differential scanning calorimetry (DSC). The thermal analysis of the chromophores shows the typical slope change of an amorphous glass transition. All heating cycles show completely amorphous behavior, without melting peaks, which can be further substantiated by the absence of X-ray diffractions in spin coated films.

The processed film can then be translated by, for example, reactive ion etching or photolithography into a waveguide structure that can be integrated with appropriate drive electronics and silica fiber transmission lines. See Dalton, L. et al., “Synthesis and Processing of Improved Organic Second-Order Nonlinear Optical Materials for Applications in Photonics”, Chemistry of Materials 7(6):1060-1081, 1995.

E-O activities and poling behaviors of the glasses can be systematically investigated with multiple variants such as loading number density, dielectric strength of films, the phenyl-perfluorophenyl interaction between molecules, and different crosslinking conditions for crosslinkable chromophores. In Table 1, the r₃₃ of multiple molecular glasses is shown. Each samples is poled at different electric fields under the optimized temperature range to quantify the field-dependence of r₃₃ values. The r₃₃ are all linearly proportional to the poling field. The solution of chromophore in 1,1,2-trichloroethane was filtered through a 0.2-μm syringe filter and spin-coated onto an ITO substrate. A typical contact poling procedure was used to pole the monolithic glassy films. The EO coefficients of poled films were measured by simple reflection method.

TABLE 1
Electro-Optic Properties of Representative Glassy Films

| | T_{g} | λₘₐₓ | Poling Field | r₃₃ |
|---|---|---|---|---|
| Chromophore | (° C.) | (nm) | (MV/cm) | (pm/V) |
| AJC135 | 45 | 719 | 0.25 | 70 |
| AJC146 | 65 | 810 | 0.4 | 120 |
| AJC168 | 65 | 812 | 0.5 | 135 |
| AJC146/AJC168 | — | 810 | 0.6 | 320 |
| MIXTURE | | | | |

The structures of AJC135, ACJ146, and AJC168 are illustrated in FIG. 1. As Table 1 indicates, mixtures of representative compounds can form glass films that have high electro-optic activity.

The compounds of the invention having modified shape through incorporating bulky side-chain group allows for the enhanced poling efficiency by isolating compounds from interacting with each other. For example, each of representative FIG. 1 compounds 1, 2, and 3 incorporate two t-butyldimethylsilyl (TBDMS) groups into the donor group through an inert alkylene (i.e., —CH₂CH₂—) linkage. Furthermore, Compound 1 includes a bridge group having four n-butyl groups (i.e., —CH₂CH₂CH₂CH₃). Compounds 1 and 2 further include acceptor groups having aryl substituents (i.e., substituted phenyl and substituted thiophenyl, respectively).

In one embodiment, the film consists essentially of a compound having a π-electron donor group electronically conjugated to a π-electron acceptor group through π-electron bridge group, the compound having the formula:

D-π₁-B-π₂-A

wherein D is a π-electron donor group, B is a π-electron bridge group, A is a π-electron acceptor group, π₁ is a π bridge electronically conjugating D to B, π₂ is a π bridge electronically conjugating B to A, wherein π₁ and π₂ may each be present or absent.

In one embodiment, the compound making up the film has one or more of the donor, bridge, and acceptor groups substituted with one or more substituents having steric bulk. Suitable substituents having steric bulk are noted above and include alkyl substituted silyl groups, substituted and unsubstituted alkyl groups, substituted and unsubstituted heteroalkyl groups, substituted and unsubstituted aryl groups, and substituted and unsubstituted heteroaryl groups.

The thickness of the film can be varied and generally has a thickness in the submicron to micron range. In one embodiment, the film has a thickness of about 1 micron.

When poled to provide acentrically-aligned compounds, the film has electro-optic activity. In one embodiment, the film has an r₃₃ value from about 50 to about 200 pm/V at 1.3 μm. In one embodiment, the film has an r₃₃ value from about 50 to about 170 pm/V at 1.3 μm.

Generally, once a compound of appropriate optical nonlinearity (μβ), optical absorption, and stability has been identified, the material can be processed into a composite material that contains acentrically-aligned compounds. As above, the processed composite can then be translated into a waveguide structure that can be integrated with appropriate drive electronics and silica fiber transmission lines.

As for the films above, in one embodiment, the compound making up the film has one or more of the donor, bridge, and acceptor groups substituted with one or more substituents having steric bulk. Suitable substituents having steric bulk are noted above and include alkyl substituted silyl groups, substituted and unsubstituted alkyl groups, substituted and unsubstituted heteroalkyl groups, substituted and unsubstituted aryl groups, and substituted and unsubstituted heteroaryl groups.

In another aspect, the present invention provides a method for forming an at least partially aligned chromophore film, comprising:

depositing a compound onto a substrate to provide a film, wherein the film consists essentially of a compound having a π-electron donor group electronically conjugated to a π-electron acceptor group through π-electron bridge group, the compound having the formula:

D-π₁-B-π₂-A

wherein D is a π-electron donor group, B is a π-electron bridge group, A is a π-electron acceptor group, π₁ is a π bridge electronically conjugating D to B, π₂ is a π bridge electronically conjugating B to A, wherein π₁ and π₂ may each be present or absent;

subjecting the film to a temperature equal to or greater than the glass transition temperature of the compound;

applying an aligning force to the film; and

reducing the temperature of the film below the glass transition temperature of the compound to provide an at least partially aligned chromophore film.

A representative embodiment of this method includes dissolving the compound in a suitable solvent, as previously described; spin-coating the solvated compound onto a suitable substrate, such as glass, semiconductor, or metal; evaporating any remaining solvent to provide a film; heating the film above the glass transition temperature of the compound, applying an electric field (i.e., poling); and cooling the film below the glass transition temperature of the compound. This is only a representative method and many variations are possible in each step. For example, a film could be deposited from the solid phase by evaporation; the film could be deposited at a temperature above the glass transition temperature of the compound, thus eliminating the heating requirement; or a magnetic or molecular (e.g., self-assembly) force could be used as an aligning force.

In one embodiment, the aligning force comprises an electric field. A representative field is between 0.2 MV/cm and 1.5 MV/cm. Corona poling can also be used as a means for electrostatic poling. Poling techniques are well known to those skilled in the art.

When a chromophore film is at least partially aligned, some of the individual chromophore molecules within the film will be non-centrosymmetrically aligned. The direction of alignment in a representative film will have a relationship to the aligning force. In one representative embodiment, the chromophore molecules will align in the direction of an electric poling field.

In one aspect the present invention provides an electro-optic device incorporating the previously described glass films. The most common of these devices are interferometers and resonators, each of which are well known and described in references cited herein and known to those skilled in the art.

To better understand the present invention, the following definitions are provided. In general, all technical and scientific terms used herein have the same meaning as commonly understood to one of skill in the art to which this invention belongs, unless clearly indicated otherwise. For clarification, listed below are definitions for certain terms used herein relating to embodiments of the present invention. These definitions apply to the terms as they are used throughout this specification, unless otherwise clearly indicated.

As used herein the singular forms “a,” “and,” and “the” include plural referents unless the context clearly dictates otherwise. For example, “a group” refers to one or more of such groups, while “a chromophore” includes a particular chromophore as well as other family members and equivalents thereof as known to those skilled in the art.

Both substituent groups and molecular moieties are sometimes represented herein with symbols (e.g., R, R¹, π, π¹, π², D, and A). When the phrase “independently at each occurrence” refers to a symbol, that symbol may represent different actual substituent groups or molecular moieties every time the symbol appears in a formula. For example, the structure below, when described by “wherein R independently at each occurrence is methyl or hydrogen,” would correspond to phenol as wells as several methyl substituted phenols including 2-methyl phenol, 3-methyl phenol, 3,4-dimethylphenol, and 2,4,6-trimethylphenol.

“Nonlinear” when used in the context of optical phenomenon pertains to second order effects. Such second order, or nonlinear, effects typically arise from a “push-pull” chromophore (i.e., a compound having the formula D-π-B-π-A).

“Electro-optic” (E-O) pertains to altering optical properties of a material by the occurrence of an electric field.

“Electronic” when used to refer to chemical structures and molecules, as opposed to electro-optic devices and components, pertains to electrons in a molecule or on an atom.

“Electric” pertains to electricity and electrical phenomena arising from applied voltages.

“Temporal stability” refers to long-term retention of a particular property. Temporal stability may be affected by any factor that modifies changes in either intermolecular order or intramolecular chemical structure.

A “π-electron bridge group” or “conjugated bridge” (represented in chemical structures by “π”) is comprised of an atom or group of atoms through which electrons can be delocalized from an electron donor group (D) to an electron acceptor (A) through the orbitals of atoms in the bridge group. Preferably, the orbitals will be p-orbitals on multiply bonded carbon atoms such as those found in alkenes, alkynes, neutral or charged aromatic rings, and neutral or charged heteroaromatic ring systems. Additionally, the orbitals can be p-orbitals on multiply bonded atoms such as boron or nitrogen or organometallic orbitals. The atoms of the bridge that contain the orbitals through which the electrons are delocalized are referred to here as the “critical atoms.” The number of critical atoms in a bridge can be a number from 1 to about 30. The critical atoms can also be substituted further with the following: “alkyl” as defined below, “aryl” as defined below, or “heteroalkyl” as defined below. One or more atoms, with the exception of hydrogen, on alkyl, aryl, or heteroalkyl substituents of critical atoms in the bridge may be bonded to atoms in other alkyl, aryl, or heteroalkyl substituents to form one or more rings.

“Donor coupling” or “π-bridge and/or donor coupling” describe the synthetic chemical step or steps known to those skilled in the art of covalently attaching a chemical group containing a donor to a selected chemical structure. The step maybe divided into multiple steps, wherein the first step covalently attaches π-bridge that is also reactive and the second step covalently attaches a donor group. Typically, the coupling involves either reacting a π-bridge or donor group containing a carbonyl with a selected chemical structure containing at least one acidic proton or reacting a π-bridge or donor group containing at least one acid proton with a selected chemical structure containing a reactive carbonyl group.

“Acceptor coupling” or “π-bridge and/or acceptor coupling” is the synthetic chemical step or steps known to those skilled in the art of covalently attaching a chemical group containing an acceptor to a selected chemical structure. The step maybe divided into multiple steps, wherein the first step covalently attaches π-bridge that is also reactive and the second step covalently attaches an acceptor group. Typically, the coupling involves either reacting a π-bridge or acceptor group containing a carbonyl with a selected chemical structure containing at least one acidic proton or reacting a π-bridge or acceptor group containing at least one acid proton with a selected chemical structure containing a reactive carbonyl group.

As used herein, “R” refers to a substituent on an atom. Unless otherwise specifically assigned, R represents any single atom or any one of the substituent groups defined below. When there is more than one R in a molecule, the “R” may independently at each occurrence refer to a single atom or any one of the substituent groups defined below.

The following definitions apply to substituent groups. A given substituent group can have a total number of carbons atoms ranging from 1 to about 200.

“Alkyl” is a saturated or unsaturated, straight or branched, cyclic or multicyclic aliphatic (i.e., non-aromatic) hydrocarbon group containing from 1 to about 30 carbons. Independently the hydrocarbon group, in various embodiments: has zero branches (i.e., is a straight chain), one branch, two branches, or more than two branches; is saturated; is unsaturated (where an unsaturated alkyl group may have one double bond, two double bonds, more than two double bonds, and/or one triple bond, two triple bonds, or more than three triple bonds); is, or includes, a cyclic stricture; is acyclic. Exemplary alkyl groups include C₁alkyl (i.e., —CH₃ (methyl)), C₂alkyl (i.e., —CH₂CH₃ (ethyl), —CH═CH₂ (ethenyl) and —C≡CH (ethynyl)) and C₃alkyl (i.e., —CH₂CH₂CH₃ (n-propyl), —CH(CH₃)₂ (i-propyl), —CH═CH—CH₃ (1-propenyl), —C≡C—CH₃ (1-propynyl), —CH₂—CH═CH₂ (2-propenyl), —CH₂—C≡CH (2-propynyl), —C(CH₃)═CH₂ (1-methylethenyl), —CH(CH₂)₂ (cyclopropyl), and adamantly. The term “alkyl” also includes groups where at least one of the hydrogens of the hydrocarbon group is substituted with at least one of the following: alkyl; “aryl” as defined below; or “heteroalkyl” as defined below. One or more of the atoms in an alkyl group, with the exception of hydrogen, can be bonded to one or more of the atoms in an adjacent alkyl group, aryl group (aryl as defined below), or heteroalkyl group (heteroalkyl as defined below) to form one or more ring.

“Aryl” is a monocyclic or polycyclic aromatic ring system or a heteroaromatic ring system (“heteroaryl”) containing from 3 to about 30 carbons. The ring system may be monocyclic or fused polycyclic (e.g., bicyclic, tricyclic, etc.). Preferred heteroatoms are nitrogen, oxygen, sulfur, and boron. In various embodiments, the monocyclic aryl ring is C5-C10, or C5-C7, or C5-C6, where these carbon numbers refer to the number of carbon atoms that form the ring system. A C6 ring system, i.e., a phenyl ring, is a preferred aryl group. A C4-S ring system (i.e., a thiophene) is another preferred aryl group. In various embodiments, the polycyclic ring is a bicyclic aryl group, where preferred bicyclic aryl groups are C8-C12, or C9-C10. A naphthyl ring, which has 10 carbon atoms, is a preferred polycyclic aryl group. The term “aryl” also includes groups where at least one of the hydrogens of the aromatic or heteroaromatic ring system is substituted further with at least one of the following: alkyl; halogen; or heteroalkyl (as defined below). One or more of the atoms in an aryl group, with the exception of hydrogen, can be bonded to one or more of the atoms in an adjacent alkyl group, aryl group, or heteroalkyl group (heteroalkyl as defined below) to form one or more rings.

“Heteroalkyl” is an alkyl group (as defined herein) wherein at least one of the carbon atoms or hydrogen atoms is replaced with a heteroatom, with the proviso that at least one carbon atom must remain in the heteroalkyl group after the replacement of carbon or hydrogen with a heteroatom. Preferred heteroatoms are nitrogen, oxygen, sulfur, silicon, and halogen. A heteroatom may, but typically does not, have the same number of valence sites as the carbon or hydrogen atom it replaces. Accordingly, when a carbon is replaced with a heteroatom, the number of hydrogens bonded to the heteroatom may need to be increased or decreased to match the number of valence sites of the heteroatom. For instance, if carbon (valence of four) is replaced with nitrogen (valence of three), then one of the hydrogens formerly attached to the replaced carbon must be deleted. Likewise, if carbon is replaced with halogen (valence of one), then three (i.e., all) of the hydrogens formerly bonded to the replaced carbon must be deleted. Examples of heteroalkyls derived from alkyls by replacement of carbon or hydrogen with heteroatoms is shown immediately below. Exemplary heteroalkyl groups are methoxy (—OCH₃), amines (—CH₂NH₂), nitriles (—CN), carboxylic acids (—CO₂H), other functional groups, and dendrons. The term “heteroalkyl” also includes groups where at least one of the hydrogens of carbon or a heteroatom of the heteroalkyl may be substituted with at least one of the following: alkyl; aryl; and heteroalkyl. One or more of the atoms in a heteroalkyl group, with the exception of hydrogen, can be bonded to one or more of the atoms in an adjacent alkyl group, aryl group, or heteroalkyl group to form one or more rings.

The substituent list that follows is not meant to limit the scope of the definitions above or the inventions described below, but rather merely contains examples of substituents within the definitions above: (1) (alkyl) —CH₃, -i-Pr, -n-Bu, -t-Bu, -i-Bu, —CH₂CH═CH₂ (allyl) —CH₂C₆H₅ (benzyl); (2) (heteroalkyl) —X₍₀₋₁₎(CH₂)₍₀₋₁₂₎(CF₂)₍₀₋₁₂₎(CH₂)₍₀₋₁₂₎CHₚZ_{q} (where X includes —O, —S, —CO₂— (ester), Z=halogen, p=0-3, q=0-3, and p+q=3) and branched isomers thereof, —X₍₀₋₁₎(CH₂)₍₀₋₁₂₎(CF₂)₍₀₋₁₂₎(CH₂)₍₀₋₁₂₎Z (where X includes —O, —S, —CO₂— (ester), Z includes —OH, —NH₂, —CO₂H and esters and amides thereof, —COCl, and —NCO) and branched isomers thereof, —OCFCF₂ (TFVE), —Si(CH₃)₃ (TMS), —Si(CH₃)₂(t-Bu) (TBDMS), —Si(C₆H₅) (TPS), —Si(C₆F₅)₃, and dendrons such as illustrated in the dendrimers discussed in Bosman et al., Chem. Rev. 1999, 99, 1665-1688; (3) (aryl)-C₆H₅ (phenyl), p-, o-, and/or m-substituted phenyl (with substituents independently selected from —CH₃, -i-Pr, -n-Bu, -t-Bu, -i-Bu, —X₍₀₋₁₎(CH₂)₍₀₋₁₂₎(CF₂)₍₀₋₁₂₎(CH₂)₍₀₋₁₂₎CHₚZ_{q} (where X includes —O, —S, —CO₂— (ester), Z=halogen, p=0-3, q=0-3, and p+q=3) and branched isomers thereof, —X₍₀₋₁₎(CH₂)₍₀₋₁₂₎(CF₂)₍₀₋₁₂₎(CH₂)₍₀₋₁₂₎Z (where X includes —O, —S, —CO₂— (ester), Z includes —OH, —NH₂, —CO₂H and esters and amides thereof, -TFVE, —COCl, and —NCO) and branched isomers thereof, —Si(CH₃)₃ (TMS), —Si(CH₃)₂(t-Bu) (TBDMS), —CH₂CH═CH₂ (allyl), and TFVE) and dendrons as illustrated in the dendrimers discussed in Bosman et al., Chem. Rev., Vol. 99, p. 1665 (1999) or U.S. Pat. No. 5,041,516.

The compounds, films, and methods described herein can be useful in a variety of electro-optic applications. In addition, these compounds, films, and methods may be applied to polymer transistors or other active or passive electronic devices, as well as OLED (organic light emitting diode) or LCD (liquid crystal display) applications.

The use of organic polymers in integrated optics and optical communication systems containing optical fibers and routers has been previously described. The compounds and films of the invention (hereinafter “materials”) may be used in place of currently used materials, such as lithium niobate, in most type of integrated optics devices, optical computing applications, and optical communication systems. For instance, the materials may be fabricated into switches, modulators, waveguides, or other electro-optical devices.

For example, in optical communication systems devices fabricated from the compounds according to the present invention may be incorporated into routers for optical communication systems or waveguides for optical communication systems or for optical switching or computing applications. Because the materials are generally less demanding than currently used materials, devices made from such polymers may be more highly integrated, as described in U.S. Pat. No. 6,049,641, which is incorporated herein by reference. Additionally, such materials may be used in periodically poled applications as well as certain displays, as described in U.S. Pat. No. 5,911,018, which is incorporated herein by reference.

Techniques to prepare components of optical communication systems from optically transmissive materials have been previously described, and may be utilized to prepare such components from materials provided by the present invention. Many articles and patents describe suitable techniques, and reference other articles and patents that describe suitable techniques, where the following articles and patents are exemplary:

L. Eldada and L. Shacklette, “Advances in Polymer Integrated Optics,” IEEE Journal of Selected Topics in Quantum Electronics, Vol. 6, No. 1, pp. 54-68 (January/February 2000); E. L. Wooten, et al. “A Review of Lithium Niobate Modulators for Fiber-Optic Communication Systems,” IEEE Journal of Selected Topics in Quantum Electronics, Vol. 6, No. 1, pp. 69-82 (January/February 2000); F. Heismann, et al. “Lithium niobate integrated optics: Selected contemporary devices and system applications,” Optical Fiber Telecommunications III B, Kaminow and Koch, eds. New York: Academic, pp. 377-462 (1997); E. Murphy, “Photonic switching,” Optical Fiber Telecommunications III B, Kaminow and Koch, eds. New York: Academic, pp. 463-501 (1997); E. Murphy, Integrated Optical Circuits and Components: Design and Applications, New York: Marcel Dekker (August 1999); L. Dalton et al., “Polymeric Electro-optic Modulators: From Chromophore Design to Integration with Semiconductor Very Large Scale Integration Electronics and Silica Fiber Optics,” Ind. Eng. Chem., Res., Vol. 38, pp. 8-33 (1999); L. Dalton et al., “From molecules to opto-chips: organic electro-optic materials,” J. Mater. Chem., Vol. 9, pp. 1905-1920 (1999); I. Liakatas et al., “Importance of intermolecular interactions in the nonlinear optical properties of poled polymers,” Applied Physics Letters, Vol. 76, No. 11, pp. 1368-1370 (13 Mar. 2000); C. Cai et al., “Donor-Acceptor-Substituted Phenylethenyl Bithiophenes: Highly Efficient and Stable Nonlinear Optical Chromophores,” Organic Letters, Vol. 1, No. 11 pp. 1847-1849 (1999); J. Razna et al., “NLO properties of polymeric Langmuir-Blodgett films of sulfonamide-substituted azobenzenes,” J. of Materials Chemistry, Vol. 9, pp. 1693-1698 (1999); K. Van den Broeck et al., “Synthesis and nonlinear optical properties of high glass transition polyimides,” Macromol. Chem. Phys. Vol. 200, pp. 2629-2635 (1999); H. Jiang, and A. K. Kakkar, “Functionalized Siloxane-Linked Polymers for Second-Order Nonlinear Optics,” Macromolecules, Vol. 31, pp. 2501-2508 (1998); A. K.-Y. Jen, “High-Performance Polyquinolines with Pendent High-Temperature Chromophores for Second-Order Nonlinear Optics,” Chem. Mater., Vol. 10, pp. 471-473 (1998); “Nonlinear Optics of Organic Molecules and Polymers,” Hari Singh Nalwa and Seizo Miyata (eds.), CRC Press (1997); Cheng Zhang, Ph.D. Dissertation, University of Southern California (1999); Galina Todorova, Ph.D. Dissertation, University of Southern California (2000); U.S. Pat. Nos. 5,272,218; 5,276,745; 5,286,872; 5,288,816; 5,290,485; 5,290,630; 5,290,824; 5,291,574; 5,298,588; 5,310,918; 5,312,565; 5,322,986; 5,326,661; 5,334,333; 5,338,481; 5,352,566; 5,354,511; 5,359,072; 5,360,582; 5,371,173; 5,371,817; 5,374,734; 5,381,507; 5,383,050; 5,384,378; 5,384,883; 5,387,629; 5,395,556; 5,397,508; 5,397,642; 5,399,664; 5,403,936; 5,405,926; 5,406,406; 5,408,009; 5,410,630; 5,414,791; 5,418,871; 5,420,172; 5,443,895; 5,434,699; 5,442,089; 5,443,758; 5,445,854; 5,447,662; 5,460,907; 5,465,310; 5,466,397; 5,467,421; 5,483,005; 5,484,550; 5,484,821; 5,500,156; 5,501,821; 5,507,974; 5,514,799; 5,514,807; 5,517,350; 5,520,968; 5,521,277; 5,526,450; 5,532,320; 5,534,201; 5,534,613; 5,535,048; 5,536,866; 5,547,705; 5,547,763; 5,557,699; 5,561,733; 5,578,251; 5,588,083; 5,594,075; 5,604,038; 5,604,292; 5,605,726; 5,612,387; 5,622,654; 5,633,337; 5,637,717; 5,649,045; 5,663,308; 5,670,090; 5,670,091; 5,670,603; 5,676,884; 5,679,763; 5,688,906; 5,693,744; 5,707,544; 5,714,304; 5,718,845; 5,726,317; 5,729,641; 5,736,592; 5,738,806; 5,741,442; 5,745,613; 5,746,949; 5,759,447; 5,764,820; 5,770,121; 5,76,374; 5,776,375; 5,777,089; 5,783,306; 5,783,649; 5,800,733; 5,804,101; 5,807,974; 5,811,507; 5,830,988; 5,831,259; 5,834,100; 5,834,575; 5,837,783; 5,844,052; 5,847,032; 5,851,424; 5,851,427; 5,856,384; 5,861,976; 5,862,276; 5,872,882; 5,881,083; 5,882,785; 5,883,259; 5,889,131; 5,892,857; 5,901,259; 5,903,330; 5,908,916; 5,930,017; 5,930,412; 5,935,491; 5,937,115; 5,937,341; 5,940,417; 5,943,154; 5,943,464; 5,948,322; 5,948,915; 5,949,943; 5,953,469; 5,959,159; 5,959,756; 5,962,658; 5,963,683; 5,966,233; 5,970,185; 5,970,186; 5,982,958; 5,982,961; 5,985,084; 5,987,202; 5,993,700; 6,001,958; 6,005,058; 6,005,707; 6,013,748; 6,017,470; 6,020,457; 6,022,671; 6,025,453; 6,026,205; 6,033,773; 6,033,774; 6,037,105; 6,041,157; 6,045,888; 6,047,095; 6,048,928; 6,051,722; 6,061,481; 6,061,487; 6,067,186; 6,072,920; 6,081,632; 6,081,634; 6,081,794; 6,086,794; 6,090,322; and 6,091,879.

The foregoing references provide instruction and guidance to fabricate waveguides from materials generally of the types described herein using approaches such as direct photolithography, reactive ion etching, excimer laser ablation, molding, conventional mask photolithography, ablative laser writing, or embossing (e.g., soft embossing). The foregoing references also disclose electron acceptors, electron donors and electron bridges that may be incorporated into the compounds of the invention.

Components of optical communication systems that may be fabricated, in whole or part, with materials according to the present invention include, without limitation, straight waveguides, bends, single-mode splitters, couplers (including directional couplers, MMI couplers, star couplers), routers, filters (including wavelength filters), switches, modulators (optical and electro-optical, e.g., birefringent modulator, the Mach-Zender interferometer, and directional and evanescent coupler), arrays (including long, high-density waveguide arrays), optical interconnects, optochips, single-mode DWDM components, and gratings. The materials described herein may be used with, for example, wafer-level processing, as applied in, for example, vertical cavity surface emitting laser (VCSEL) and CMOS technologies.

In many applications, the materials described herein may be used in lieu of lithium niobate, gallium arsenide, and other inorganic materials that currently find use as light-transmissive materials in optical communication systems.

The materials described herein may be used in telecommunication, data communication, signal processing, information processing, and radar system devices and thus may be used in communication methods relying, at least in part, on the optical transmission of information. Thus, a method according to the present invention may include communicating by transmitting information with light, where the light is transmitted at least in part through a material including a compound of the invention.

The materials of the present invention can be incorporated into various electro-optical devices. Accordingly, in another aspect, the invention provides electro-optic devices including the following:

an electro-optical device comprising a material of the present invention;

a waveguide comprising a material of the present invention;

an optical switch comprising a material of the present invention;

an optical modulator comprising a material of the present invention;

an optical coupler comprising a material of the present invention;

an optical router comprising a material of the present invention;

a communications system comprising a material of the present invention;

a method of data transmission comprising transmitting light through or via a material of the present invention;

a method of telecommunication comprising transmitting light through or via a material of the present invention;

a method of transmitting light comprising directing light through or via a material of the present invention;

a method of routing light through an optical system comprising transmitting light through or via a material of the present invention;

an interferometric optical modulator or switch, comprising: (1) an input waveguide; (2) an output waveguide; (3) a first leg having a first end and a second end, the first leg being coupled to the input waveguide at the first end and to the output waveguide at the second end; and 4) and a second leg having a first end and a second end, the second leg being coupled to the input waveguide at the first end and to the output waveguide at the second end, wherein at least one of the first and second legs includes a material of the present invention;

an optical modulator or switch, comprising: (1) an input; (2) an output; (3) a first waveguide extending between the input and output; and (4) a second waveguide aligned to the first waveguide and positioned for evanescent coupling to the first waveguide; wherein at least one of the first and second legs includes a material of the present invention. The modulator or switch may further including an electrode positioned to produce an electric field across the first or second waveguide;

an optical router comprising a plurality of switches, wherein each switch includes: (1) an input; (2) an output; (3) a first waveguide extending between the input and output; and (4) a second waveguide aligned to the first waveguide and positioned for evanescent coupling to the first waveguide; wherein at least one of the first and second legs includes a material of the present invention. The plurality of switches may optionally be arranged in an array of rows and columns.

In summary, the present invention provides highly efficient nonlinear optical (NLO) compounds with nanoengineered structural modifications can form high quality, micron-thick films with good thermal and dielectric properties. By moderate poling fields from about 0.15 to about 0.60 MV/cm, poled films (e.g., monolithic glasses or their composites) exhibit very large electro-optic (E-O) coefficients (i.e., up to 150-310 pm/V) at the telecommunication wavelengths of 1310 and 1550 nm. This type of organic glass eliminates the necessity of, or complication brought by, using polymers and/or dendrimers at the matrices for common bulky organic NLO materials. The monolithic glasses of the invention overcome the limited compatibility, mismatched dielectric property and conductivity, and different phase transition behavior between chromophoric compounds and matrices in typical E-O polymers or dendrimers. Main advantages of the monolithic glasses of the invention include simplified material formulation and processing, improved poling efficiency and performance reproducibility, and two to three (2-3×) times higher E-O activities than their polymer or dendrimer counterparts, creating a new series of high-performance E-O and photo-refractive materials for photonics and opto-electronics.

Each reference cited in the application, including citations to literature and patent documents, is expressly incorporated herein by reference in its entirety.

The following examples are provided for the purpose of illustrating, not limiting, the invention.

### EXAMPLES

### Example 1

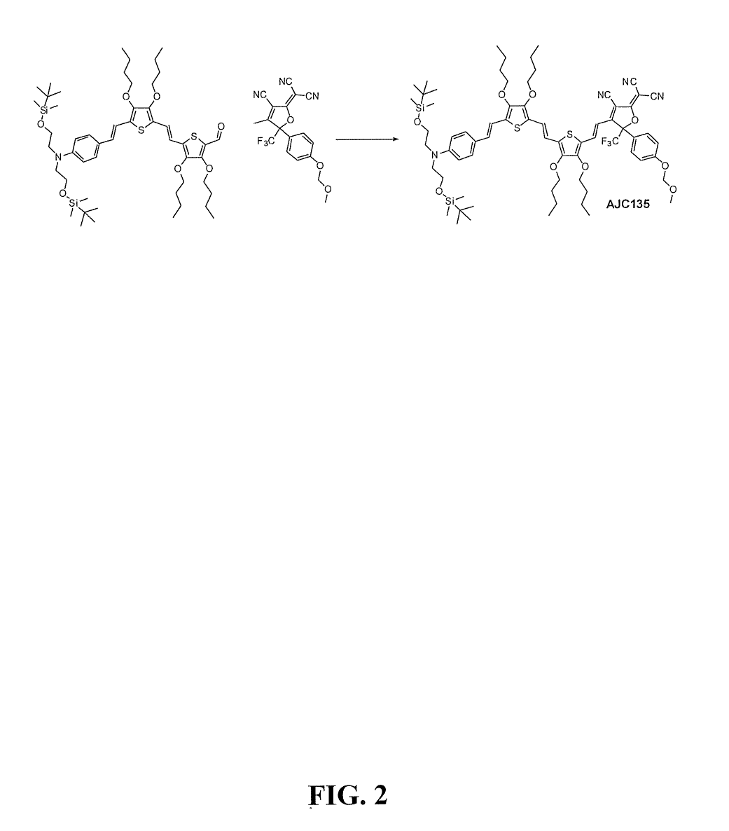

In this example, the preparation of a representative compound of the invention is described below and illustrated schematically in FIG. 2.

Compound 1 was prepared from a donor-bridge and an acceptor developed as described below. The donor-bridge was prepared as described in U.S. Pat. No. 6,750,603, incorporated herein by reference in its entirety. The donor-bridge aldehyde (0.753 g, 0.8 mmol) and acceptor (0.375 g, 1.0 mmol) were dissolved in anhydrous ethanol (1.0 mL) and the mixture was stilled at around 50° C. for 4 hours. The crude product was purified by flash chromatography and recrystallization in methanol/methylene dichloride several times to afford Compound 1 as dark solid (yield: 48%).

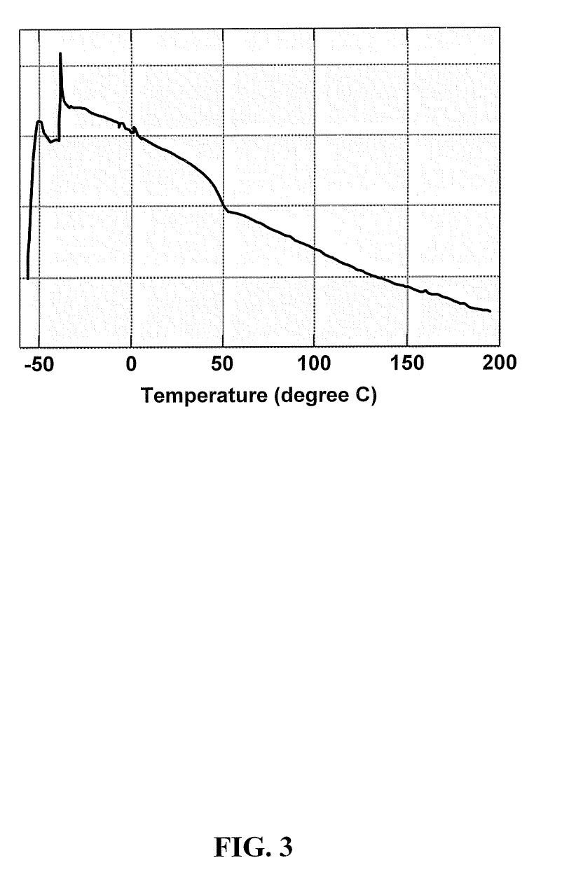

¹H NMR data (CDCl₃, TMS): δ=7.97 (d, 1H, CH═), 7.45 (d, 2H, Ar), 7.35 (d, 3H, Ar+CH═), 7.28 (d, 1H, CH═), 7.16 (d, 2H, Ar) 7.05 (d, 1H, CH═), 6.89 (d+d, 2H, CH═), 6.73 (d, 2H, Ar), 6.56 (d, 2H, CH═), 5.24 (s, 2H, OCH₂O), 4.20 (t+t, 4H, CH₂O), 4.07 (t, 2H, CH₂O), 4.01 (t, 2H, CH₂O), 3.80 (t, 4H, CH₂O), 3.57 (t, 4H, NCH₂), 3.51 (s, 3H, CH₃O), 1.79 (m, 6H, CH₂ of butyl), 1.68 (m, 2H, CH₂ of butyl), 1.57 (m, 6H, CH₂ of butyl), 1.41 (m, 2H, CH₂ of butyl), 1.0 (m, 9H, CH₃ of butyl), 0.97 (t, 3H, CH₃ of butyl), 0.92 (s, 18H, CH₃ of t-butyl group on TBDMS), 0.07 (s, 12H, CH₃ of TBDMS). Glass transition temperature by DSC: T_{g}=47° C. The differential scanning calorimetry (DSC) curve for Compound 1 is illustrated in FIG. 3.

### Example 2

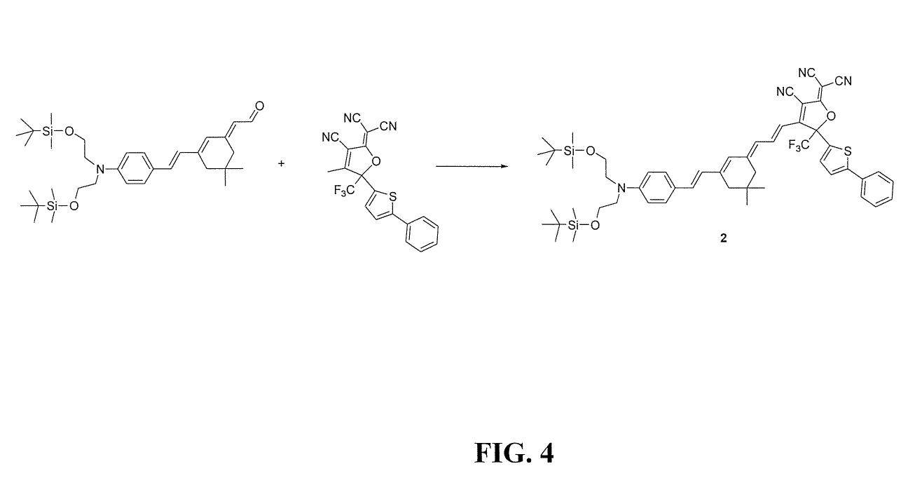

In this example, the preparation of a representative compound of the invention is described and illustrated schematically in FIG. 4. The final product is composed of a known donor/bridge and the acceptor 4 synthesized by the following reactions:

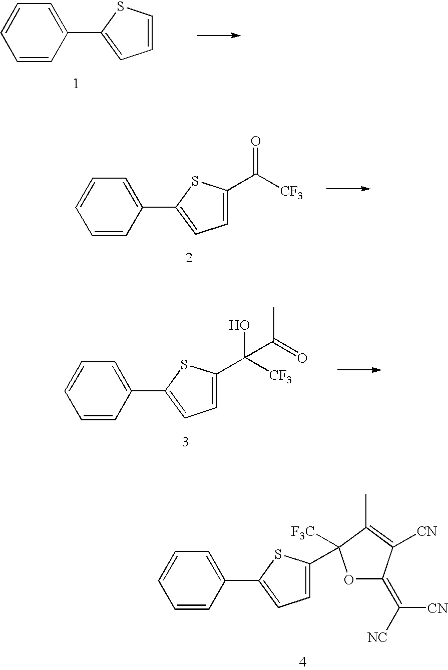

Compound 2: BuLi (2.5M, 42 ml) was added to compound 1 in THF (100 ml) at −78° C. After this addition, the temperature was allowed to rise to −40° C. The temperature was cooled to −78° C., then CF₃CO₂Et (3.62 g) was added slowly to the mixture. The reaction was kept stirring overnight. The reaction was quenched with brine and organic layer was separated and dried over Na₂SO₄. After removal of solvent, the product was purified by silica gel column to give a yellowish leaf-like crystals (12.3 g). ¹H-NMR, (CDCl₃, TMS): δ=8.1 (d, 2H, phenyl), 7.45 (m, 3H, phenyl), 7.43 (dd, 1H, thiophene), 7.39 (dd, 1H, thiophene), 7.38 (dd, 1H, thiophene).

Compound 3 was synthesized following a similar procedure for a ketol precursor as found in Liu, Sen; Haller, Marnie A.; Ma, Hong; Dalton, Larry R.; Jang, Sei-Hum; Jen, Alex K.-Y. Adv. Mater. (2003), 15(7-8), 603-607 and He, Mingqian; Leslie, Thomas M.; Sinicropi, Jolm A. Chem. Mater. (2002), 14(5), 2393-2400. ¹H-NMR (CDCl₃, TMS): δ=7.60 (d, 2H, phenyl), 7.41 (m, 2H, thiophene+phenyl) 7.34 (t, 2H, phenyl), 7.28 (d, 1H, thiophene), 5.25 (s, 1H, OH), 2.54 (s, 3H, CH₃). Yield: 37%.

Acceptor 4: a mixture of compound 3 (0.60 g), malonitrile (0.33 g), sodium ethoxide in ethanol (1M, 0.1 mL) and ethanol (0.9 mL) in a 25 ml flask with a magnetic stir bar was heated with 15-25 W microwave at 95° C. for 40 min. to give acceptor 4 (119 mg) after column chromatography. ¹H-NMR (CDCl₃, TMS): δ=7.60 (m, 2H, thiophene), 7.43 (m, 3H, thiophene) 7.33 (m, 2H, thiophene), 2.58 (s, 3H, CH₃).

AJC-146 (FIG. 4, compound 2) was synthesized (yield: 73%) from a known donor-bridge aldehyde and acceptor 4.

Compound 2 was prepared by the following procedure. To 0.5 mL of dry ethanol was added 0.161 g (0.270 mmol) of bridge aldehyde and 0.108 g (0.272 mmol) of acceptor. The mixture was heated to 40° C. under nitrogen atmosphere for two and a half hours. The crude product was purified through chromatography on silica gel with the eluent of 5-10% ethyl acetate in hexane to afford Compound 2 as dark powder (0.191 g, yield: 73%), which was recrystallized in methanol twice prior to use.

¹H NMR data (CDCl₃, TMS): δ=8.27 (d, 1H, CH═), 7.62 (d, 1H, thiophene), 7.41 (d, 3H, phenylene and CH═), 7.29 (m, 1H para-phenylene), 6.96 (d, 1H, CH═), 6.83 (d, 1H, CH═), 6.72 (d, 2H, phenylene), 6.43 (d, 1H, thiophene), 6.38 (d, 1H, CH═), 3.80 (t, 4H, CH₂O), 3.59 (t, 4H, CH₂N), 2.41 (m, 4H, CH₂ of cyclohexylene), 1.03 (m, 6H, CH₃ oil cyclohexylene), 0.90 (m, 18H, CH₃ of t-butyl), 0.04 (s, 12H, CH₃ of TBDMS). Glass transition temperature by DSC: T_{g}=65° C.

### Example 3

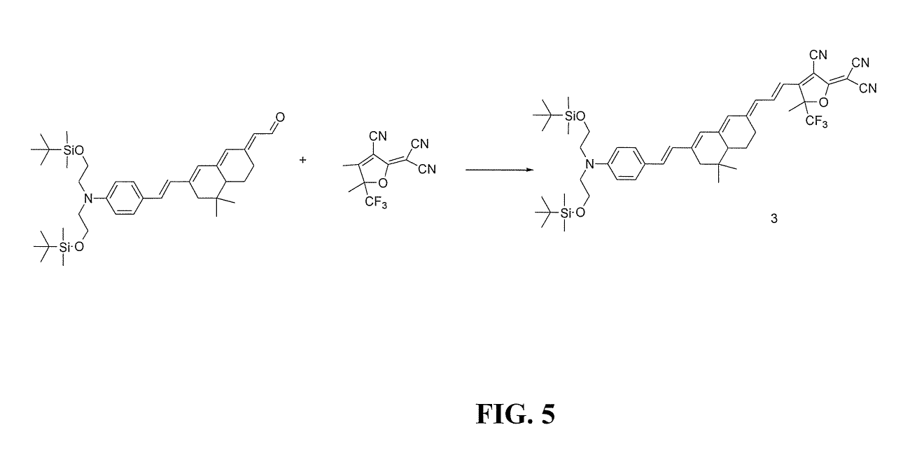

In this example, the preparation of a representative compound of the invention is described and illustrated schematically in FIG. 5.

Compound 3 was prepared by the following procedure. To 1.0 mL of dry ethanol was added 0.217 g (0.341 mmol) of bridge aldehyde (as detailed in Dalton, L. R. Advances in Polymer Science Vol. 158, 1, 2002) and 0.0888 g (0.35 mmol) of acceptor. The mixture was heated to room temperature under nitrogen atmosphere for 100 mins. The crude product was purified through chromatography on silica gel to afford Compound 3 as dark powder (0.160 g, yield: 53%), which has been recrystallized in methanol twice prior to use.

¹H NMR data (CDCl₃, TMS): δ=8.42 (d, 1H, CH═), 7.36 (d, 1H, phenylene), 6.80 (m, 2H, CH═), 6.69 (d, 2H, phenylene), 6.35 (d, 3H, CH═), 6.14 (d, 1H, CH═), 3.79 (t, 4H, OCH₂), 3.58 (t, 4H, CH₂N), 3.18 (d, 1H, CH on fused ring), 2.57-2.39 (m, 6H, CH₂ on fused cyclohexylene), 1.84 (s, 3H, CH₃), 1.20 (m, 4H, CH₃ on ring), 0.90 (m, 18H, CH₃ of t-butyl), 0.05 (m, 12H, CH₃ of CH₃Si). Glass transition temperature by DSC: T_{g}=110° C.

### Example 4

A range of triarylamino donors in two series of nonlinear optical chromophores A and B were systematically investigated and are illustrated in FIG. 11 (see A1-A4 and B1-B4). The π-donor strength of the triphenylamino group can be tuned and enhanced by incorporation of donating alkoxy groups para to the nitrogen on the phenyl rings. Each of these chromophores showed amorphous behavior by DSC and formed monolithic thin films by themselves. With the aid of two donating methoxy substituents on the aromatic donor, both A4 and B3 exhibit significantly improved thermal stability while maintaining a reasonably high nonlinearity. A shape modification with a perfluoro-aromatic dendron anchored at the donor in B4 further improved the thermal stability and poling efficiency, leading to the highest T_{d} of 237° C. and the largest E-O coefficient (r₃₃) of 169 pm/V.

An experimental method for making the representative family of chromophores A1-A4 is illustrated in FIG. 12. The reagents and conditions for FIG. 12 are as follows: (a) 1 equiv of POCl₃ and DMF, 1,2-dichloroethane, rt 24 h, then H₂O; (b) DIBAL-H, THF, −78° C., 2 h; (c) phosphonate, 1.2 equiv of t-BuOK., THF, 0° C. to reflux, 24 h; (d) n-BuLi, THF, −78° C., 1 h then DMF, −78° C. to rt; (e) aldehyde and 1.1 equiv of acceptor 5, ethanol, 50° C., 2 h.

An experimental method for making the representative family of chromophores B1-B4 is illustrated in FIG. 13. Reagents and conditions: (a) 1 equiv of Na in ethanol, then isophorone and aldehyde 2, THF, 60° C., 24 hr; (b) 2.5 equiv of NaH and 2.5 equiv of (C₃H₇O)₂(CH₂CN)P═O in THF then 6, reflux, 18 hr; (c) 1.5 equiv of DIBAL, —H, toluene, −78° C., 2 h then HCl (aq); (d) 6M HCl, THF, reflux, 8 hr; (e) aldehyde 8 and 1.1 equiv of acceptor 5, ethanol 50° C., 2 hr; (f) 10 equiv of 3,5-bis(trifluoromethyl)-benzoyl chloride in THF, reflux, 12 h.

While illustrative embodiments have been illustrated and described, it will be appreciated that various changes can be made therein without departing from the spirit and scope of the invention.

The embodiments of the invention in which an exclusive property or privilege is claimed are defined as follows:

1. A film, consisting essentially of a compound having a π-electron donor group electronically conjugated to a π-electron acceptor group through π-electron bridge group, the compound having the formula:
D-π₁-B-π₂-A
wherein D is a π-electron donor group, B is a π-electron bridge group, A is a π-electron acceptor group, π₁ is a π bridge electronically conjugating D to B, π₂ is a π bridge electronically conjugating B to A, wherein π₁ and π₂ may each be present or absent.

2. The film of claim 1, wherein one or more of the donor, bridge, and acceptor groups substituted with one or more substituents having steric bulk.

3. The film of claim 2, wherein the substituent having steric bulk is selected from the group consisting of an alkyl substituted silyl group, a substituted or unsubstituted alkyl group, a substituted or unsubstituted heteroalkyl group, a substituted or unsubstituted aryl group, and a substituted or unsubstituted heteroaryl group.

4. The film of claim 3, wherein the alkyl substituted silyl group is a t-butyldimethylsilyl group.

5. The film of claim 3, wherein the alkyl group is a branched or straight chain C4 alkyl group.

6. The film of claim 3, wherein the aryl group is a substituted phenyl group.

7. The film of claim 3, wherein the heteroaryl group is a substituted thiophene group.

8. The film of claim 1 having a thickness of about 1 micron.

9. The film of claim 1 having an r₃₃ value from about 50 to about 200 pm/V at 1.3 μm.

10. A method for forming an at least partially aligned chromophore film, comprising:
(a) depositing a compound onto a substrate to provide a film, wherein the film consists essentially of a compound, having a π-electron donor group electronically conjugated to a π-electron acceptor group through π-electron bridge group, the compound having the formula:
D-π₁-B-π₂-A
wherein D is a π-electron donor group, B is aπ-electron bridge group, A is a π-electron acceptor group, π₁ is a π bridge electronically conjugating D to B, π₂ is a π bridge electronically conjugating B to A, wherein π₁ and π₂ may each be present or absent;
(b) subjecting the film to a temperature equal to or greater than the glass transition temperature of the compound;
(c) applying an aligning force to the film; and
(d) reducing the temperature of the film below the glass transition temperature of the compound to provide a film comprising an at least partially aligned chromophore film.

11. The method of claim 10, wherein the chromophore aligning force comprises an electric field.

12. An electro-optic device, comprising a film consisting essentially of a compound having a π-electron donor group electronically conjugated to a π-electron acceptor group through π-electron bridge group, the compound having the formula:
D-π₁-B-π₂-A
wherein D is a π-electron donor group, B is a π-electron bridge group, A is a π-electron acceptor group, π₁ is a π bridge electronically conjugating D to B, π₂ is a π bridge electronically conjugating B to A, wherein π₁ and π₂ may each be present or absent, and wherein the compound has the ability to form by itself a film having electro-optic activity.

13. The device of claim 12, wherein the electro-optic device comprises an interferometer.

14. The device of claim 12, wherein the electro-optic device comprises a resonator.
